(19) United States
(12) Patent Application Publication
(10) Pub. No.: US 2026/0321389 A1
(43) Pub. Date: Oct. 15, 2026

(54) METHOD FOR ADAPTIVE RADIOTHERAPY TREATMENT DELIVERY

(71) Applicant: Elekta Limited, Crawley, West Sussex (GB)

(72) Inventor: Raymond DALFSEN, Crawley, West Sussex (GB)

(21) Appl. No.: 19/483,479

(22) PCT Filed: May 10, 2024
(86) PCT No.: PCT/EP2024/062972

§ 371 Date: Nov. 11, 2025

(30) Foreign Application Priority Data
May 11, 2023 (GB) 2307019.6
May 12, 2023 (GB) 2307143.4

(51) Int. Cl. G16H 20/40; A61N 5/10

(52) U.S. Cl. CPC G16H 20/40; A61N 5/1038; A61N 5/1039; A61N 5/1048; A61N 2005/1074

(57) ABSTRACT

Computer-implemented methods, systems, and computer-readable media are disclosed for selecting a first workflow path, wherein the first workflow path comprises replacing at least part of a scheduled radiotherapy treatment plan with at least part of a previously-generated radiotherapy treatment plan. The previously-generated radiotherapy treatment plan is identified based on a determined structural characteristic.

This disclosure relates to radiotherapy workflows, and in particular to a system and method for use in a radiotherapy workflow.

### BACKGROUND

Radiotherapy can be described as the use of ionising radiation, such as X-rays, to treat a human or animal body. Radiotherapy is commonly used to treat tumours within the body of a patient or subject. In such treatments, ionising radiation is used to irradiate, and thus destroy or damage, cells which form part of the tumour. In radiotherapy treatment, it is desirable to deliver a prescribed dose of radiation to a target region, e.g. a tumour, of a patient and to limit irradiation of other parts of the patient, e.g. healthy tissue and organs at risk (OARs).

The treatment planning procedure typically involves obtaining one or more medical images, such as a CT image of the patient, and segmenting them to identify the target region and OARs near the target region. The segmentation process can be performed manually or using auto-segmentation techniques. The clinician determines radiation treatment parameters, for example by prescribing a radiation dose to be delivered to the target region and maximum doses which can safely be delivered to the various OARs. The treatment planning procedure may then involve optimizing various radiation delivery variables to meet the prescribed radiation treatment parameters, for example determining the number of sessions (or ‘fractions’) over which radiotherapy should be conducted, the angles at which the radiation beam should be applied during each fraction, at what beam energy, the duration of application of the radiation beams at these angles, and the beam shape(s) at each angle of delivery. The clinician can be assisted by software during some or all of these steps. This aspect of treatment planning, conducted in advance of the patient's treatment (and sometimes between treatment fractions if there are gradual changes, making the need for a plan adaptation foreseeable), can be described as “offline” treatment planning as part of an offline adaptive treatment workflow.

However, the characteristics of the tumour, such as its shape and size, may change over the course of the multiple fractions of treatment. Similarly, other patient information and characteristics of the patient's anatomy can change between fractions, in both cases in a less foreseeable way. Accordingly, “online” adaptive radiotherapy techniques may be used to update and re-optimise the radiotherapy treatment plan and/or delivery variables immediately prior to the patient's treatment. According to online adaptive radiotherapy techniques, the patient is imaged again immediately prior to treatment, and the treatment plan for that day's fraction may be adjusted and/or reoptimized according to the latest available medical images. Online adaptive radiation therapy techniques, as part of an online treatment workflow, therefore allow inter-fraction anatomical changes to be taken into account.

Both traditional offline and online adaptive treatment workflows have advantages, and selection of which of the two available workflows should be used (offline or online ART) depends on a number of factors including the available time, the clinical goal(s), available expertise and technology, and available resources. Currently available online adaptive treatment workflows require a lot of manual steps and decisions, each of which require appropriate skills and clinical experience at the point of treatment delivery. In other words, current online adaptive workflows require a high level of expertise and experience to use effectively, and experienced clinicians must be present on-site for the workflow to operate, which is time and resource intensive. In addition, currently available treatment workflows require upfront decisions by clinicians on the appropriate care path for the patient. In other words, clinicians must decide before the start of treatment if a patient will experience the ‘standard’ (or ‘traditional’) IGRT workflow, or the online adaptive workflow.

The present invention seeks to address these and other disadvantages encountered in the prior art by providing an improved method and system for use in a radiotherapy workflow.

### SUMMARY

An invention is set out in the independent claims. Optional features are set out in the dependent claims.

### FIGURES

Specific examples are now described, by way of example only, with reference to the drawings, in which:

FIG. 1 shows a radiotherapy device or apparatus according to the present disclosure;

FIG. 2 shows a radiotherapy treatment workflow according to the present disclosure;

FIG. 3 shows a computer-implemented method for use in a radiotherapy workflow according to the present disclosure;

FIG. 4 shows an example portion of a radiotherapy user interface according to the present disclosure;

FIG. 5 shows a further example portion of a radiotherapy user interface according to the present disclosure;

FIG. 6 shows a further example portion of a radiotherapy user interface according to the present disclosure;

FIG. 7 shows a further example portion of a radiotherapy user interface according to the present disclosure;

FIG. 8 shows a block diagram of one implementation of a radiotherapy system;

FIG. 9 shows a computer readable medium or, more generally, a computer program product;

FIG. 10 shows a method according to the present disclosure;

FIG. 11 shows a method according to the present disclosure; and

FIG. 12 shows a computer-implemented method for use in a radiotherapy workflow according to the present disclosure.

### OVERVIEW

In a non-limiting overview provided to aid understanding, a computer-implemented method, and system, is provided that is capable of changing from a standard pre-determined image-guided radiation therapy (IGRT) workflow to an online adaptive radiotherapy workflow on a fraction-by-fraction basis.

Using this method or/and system, a clinician no longer needs to decide whether or not to use online adaptive radiotherapy upfront when determining the initial treatment plan. Furthermore, the method and/or system can be implemented without requiring that clinicians are present on site during treatment delivery to make a manual assessment of any deviation from the original treatment plan. That allows for fewer and/or less-experienced/skilled medical staff to deliver online adaptive radiotherapy at that particular session, providing improvements to the cost, efficiency, and/or resourcing of radiotherapy without compromising on patient safety or treatment efficacy.

Yet furthermore, by providing a visual indicator of whether delivering the scheduled radiotherapy treatment plan with the ROI at the second position would meet the chosen treatment plan quality criteria, a user may very quickly determine which workflow to follow in the interests of patient safety or to improve treatment efficacy, again saving time in delivering treatment without compromising treatment quality. This is particularly important for situations in which the patient may be uncomfortable or in pain. It is generally desirable to reduce the time the patient spends on the patient support system for reasons of patient comfort and convenience, making it undesirable to manually assess or re-assess the treatment plan in every case.

In another non-limited overview provided to aid understanding, also provided herein are a computer-implemented method, and system, that are capable of selecting a first workflow path on a fraction-by-fraction basis, wherein the first workflow path comprises replacing at least part of a scheduled radiotherapy treatment plan with at least part of a previously-generated radiotherapy treatment plan, the previously-generated radiotherapy treatment plan having been identified based on a determined structural characteristic of an obtained image of a patient. Using this method and/or system, the time required to change and/or adapt a scheduled radiotherapy treatment plan can be reduced while still providing the improved accuracy that adaptive radiotherapy can provide.

Further examples and advantages are set out below.

### DETAILED DESCRIPTION

Disclosed herein are systems, devices, methods and apparatuses relating to radiotherapy. With linear accelerator-based radiotherapy devices being highly complex and having many inter-related parts, the terms “system”, “device”, “apparatus”, and “machine” may all be applied interchangeably to describe the radiotherapy device as a whole, or collections of components of the radiotherapy device.

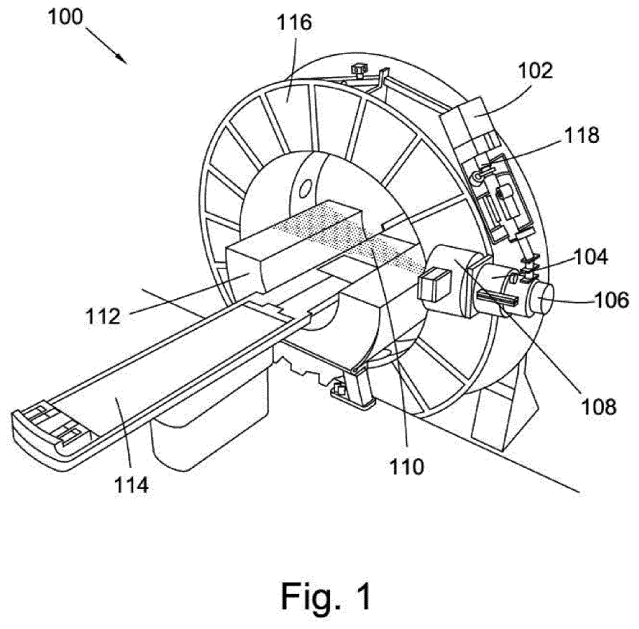

FIG. 1 shows an exemplary radiotherapy (RT) device 100. The device and its constituent components will be well known to the skilled person but is described here generally for the purpose of providing useful accompanying information for the present disclosure. The radiotherapy device 100 is based on a linear accelerator (linac).

The device shown in FIG. 1 combines magnetic resonance (MR) imaging capability with a linac-based radiotherapy capability, and is known as an MR-linac device. MR-linacs are particularly well-suited for delivery of adaptive treatment, since MR images may be taken immediately prior to or during treatment. The device and its constituent components will be described generally for the purpose of providing useful accompanying information for the present invention. The device depicted in FIG. 1 is in accordance with the present disclosure and is suitable for use with the disclosed systems and apparatuses. However, the present disclosure may be implemented in any radiotherapy device, for example, a linac-based radiotherapy device with CBCT imaging capability.

The device 100 in FIG. 1 comprises both MR imaging apparatus 112 and radiotherapy (RT) apparatus which may comprise a linac device. The MR imaging apparatus 112 is shown in cross-section in the diagram. In operation, the MR scanner produces MR images of the patient, and the linac device produces and shapes a beam of radiation and directs it toward a target region within a patient's body in accordance with a radiotherapy treatment plan. The depicted device does not have the usual ‘housing’ which would cover the MR imaging apparatus 112 and RT apparatus in a commercial setting such as a hospital.

The MR-linac device depicted in FIG. 1 comprises a source of radiofrequency waves 102, a waveguide 104, a source of electrons 106, a source of radiation 106, a collimator 108 such as a multi-leaf collimator configured to collimate and shape the beam, MR imaging apparatus 112, and a patient support surface 114. In use, the device would also comprise a housing (not shown) which, together with the ring-shaped gantry, defines a bore. The moveable support surface 114 can be used to move a patient, or other subject, into the bore when an MR scan and/or when radiotherapy is to commence. The MR imaging apparatus 112, RT apparatus, and a subject support surface actuator are communicatively coupled to a controller or processor. The controller is also communicatively coupled to a memory device comprising computer-executable instructions which may be executed by the controller.

The RT apparatus comprises a source of radiation and a radiation detector (not shown). Typically, the radiation detector is positioned diametrically opposed to the radiation source. The radiation detector is suitable for, and configured to, produce radiation intensity data. In particular, the radiation detector is positioned and configured to detect the intensity of radiation which has passed through the subject. The radiation detector may also be described as radiation detecting means, and may form part of a portal imaging system.

The radiation source may comprise a beam generation system. For a linac, the beam generation system may comprise a source of RF energy 102, an electron gun 106, and a waveguide 104. The radiation source is attached to the rotatable gantry 116 so as to rotate with the gantry 116. In this way, the radiation source is rotatable around the patient so that the treatment beam 110 can be applied from different angles around the gantry 116. In a preferred implementation, the gantry is continuously rotatable. In other words, the gantry can be rotated by 360 degrees around the patient, and in fact can continue to be rotated past 360 degrees. The gantry may be ring-shaped. In other words, the gantry may be a ring-gantry.

The source 102 of radiofrequency waves, such as a magnetron, is configured to produce radiofrequency waves. The source 102 of radiofrequency waves is coupled to the waveguide 104 via circulator 118, and is configured to pulse radiofrequency waves into the waveguide 104. Radiofrequency waves may pass from the source 102 of radiofrequency waves through an RF input window and into an RF input connecting pipe or tube. A source of electrons 106, such as an electron gun, is also coupled to the waveguide 104 and is configured to inject electrons into the waveguide 104. In the electron gun 106, electrons are thermionically emitted from a cathode filament as the filament is heated. The temperature of the filament controls the number of electrons injected. The injection of electrons into the waveguide 104 is synchronised with the pumping of the radiofrequency waves into the waveguide 104. The design and operation of the radiofrequency wave source 102, electron source and the waveguide 104 is such that the radiofrequency waves accelerate the electrons to very high energies as the electrons propagate through the waveguide 104.

The design of the waveguide 104 depends on whether the linac accelerates the electrons using a standing wave or travelling wave, though the waveguide typically comprises a series of cells or cavities, each cavity connected by a hole or ‘iris’ through which the electron beam may pass. The cavities are coupled in order that a suitable electric field pattern is produced which accelerates electrons propagating through the waveguide 104. As the electrons are accelerated in the waveguide 104, the electron beam path is controlled by a suitable arrangement of steering magnets, or steering coils, which surround the waveguide 104. The arrangement of steering magnets may comprise, for example, two sets of quadrupole magnets.

Once the electrons have been accelerated, they may pass into a flight tube. The flight tube may be connected to the waveguide by a connecting tube. This connecting tube or connecting structure may be called a drift tube. The electrons travel toward a heavy metal target which may comprise, for example, tungsten. Whilst the electrons travel through the flight tube, an arrangement of focusing magnets act to direct and focus the beam on the target.

To ensure that propagation of the electrons is not impeded as the electron beam travels toward the target, the waveguide 104 is evacuated using a vacuum system comprising a vacuum pump or an arrangement of vacuum pumps. The pump system is capable of producing ultra-high vacuum (UHV) conditions in the waveguide 104 and in the flight tube. The vacuum system also ensures UHV conditions in the electron gun. Electrons can be accelerated to speeds approaching the speed of light in the evacuated waveguide 104.

The source of radiation is configured to direct a beam 110 of therapeutic radiation toward a patient positioned on the patient support surface 114. The source of radiation may comprise a heavy metal target toward which the high energy electrons exiting the waveguide are directed. When the electrons strike the target, X-rays are produced in a variety of directions. A primary collimator may block X-rays travelling in certain directions and pass only forward travelling X-rays to produce a treatment beam 110. The X-rays may be filtered and may pass through one or more ion chambers for dose measuring. The beam can be shaped in various ways by beam-shaping apparatus, for example by using a multi-leaf collimator 108, before it passes into the patient as part of radiotherapy treatment.

In some implementations, the source of radiation is configured to emit either an X-ray beam or an electron particle beam. Such implementations allow the device to provide electron beam therapy, i.e. a type of external beam therapy where electrons, rather than X-rays, are directed toward the target region. It is possible to ‘swap’ between a first mode in which X-rays are emitted and a second mode in which electrons are emitted by adjusting the components of the linac. In essence, it is possible to swap between the first and second mode by moving the heavy metal target in or out of the electron beam path and replacing it with a so-called ‘electron window’. The electron window is substantially transparent to electrons and allows electrons to exit the flight tube.

The subject or patient support surface 114 is configured to move between a first position substantially outside the bore, and a second position substantially inside the bore. In the first position, a patient or subject can mount the patient support surface. The support surface 114, and patient, can then be moved inside the bore, to the second position, in order for the patient to be imaged by the MR imaging apparatus 112 and/or imaged or treated using the RT apparatus. The movement of the patient support surface is effected and controlled by a subject support surface actuator, which may be described as an actuation mechanism. The actuation mechanism is configured to move the subject support surface in a direction parallel to, and defined by, the central axis of the bore. The terms subject and patient are used interchangeably herein such that the subject support surface can also be described as a patient support surface. The subject support surface may also be referred to as a moveable or adjustable couch or table.

The radiotherapy apparatus/device depicted in FIG. 1 also comprises MR imaging apparatus 112. The MR imaging apparatus 112 is configured to obtain images of a patient or subject positioned, i.e. located, on the subject support surface 114. The MR imaging apparatus 112 may also be referred to as the MR imager. The MR imaging apparatus 112 may be a conventional MR imaging apparatus operating in a known manner to obtain MR data, for example MR images. The skilled person will appreciate that such a MR imaging apparatus 112 may comprise a primary magnet, one or more gradient coils, one or more receive coils, and an RF pulse applicator. The operation of the MR imaging apparatus is controlled by the controller.

The controller is a computer, processor, or other processing apparatus. The controller may be formed by several discrete processors; for example, the controller may comprise an MR imaging apparatus processor, which controls the MR imaging apparatus 110; an RT apparatus processor, which controls the operation of the RT apparatus; and a subject support surface processor which controls the operation and actuation of the subject support surface. The controller is communicatively coupled to a memory, e.g. a computer readable medium.

The linac device also comprises several other components and systems as will be understood by the skilled person. For example, in order to ensure the linac does not leak radiation, appropriate shielding is also provided.

In conventional approaches to radiotherapy, a dose for the target/tumour is determined and prescribed, and that dose is then delivered in fractions across a number of sessions, which reduces the effects of radiation that is delivered to healthy tissue of the patient. Accordingly, a radiotherapy treatment plan is determined and scheduled by clinicians in advance of beginning treatment. As set out above, that treatment plan may be determined and scheduled with traditional IGRT or online adaptive workflows. In addition, an off-line plan adjustment may be scheduled. Such a determination is conventionally made at the initial planning stage prior to any of the fractional radiotherapy sessions, in part due to time and/or resource considerations.

In methods and/or systems disclosed herein, in an initial planning stage before beginning any treatment, a scheduled radiotherapy treatment plan for delivering a prescribed dose is determined. The scheduled radiotherapy treatment plan may comprise a treatment schedule component, e.g. how the dose is divided into fractions and when those fractions are delivered, and a radiotherapy delivery plan, which determines particular system variables or parameters for delivering a particular dose fraction to a particular site, such as suitable beam shaping component positions or configurations, beam angles, beam energies, or/and duration of exposure. In addition, at least one treatment plan quality criterion may be pre-defined during the initial planning stage, which may be considered as the most important criterion or criteria for evaluating the plan, and further details of which are provided below. The at least one treatment plan quality criterion may correspond to one of, or a subset of, the parameters or variables upon which the scheduled radiotherapy treatment plan is based, such as a particular maximum value for the dose delivered to a particular OAR. Unlike conventional approaches, the methods and/or systems disclosed herein enable the radiotherapy workflow to be changed on a fraction-to-fraction basis.

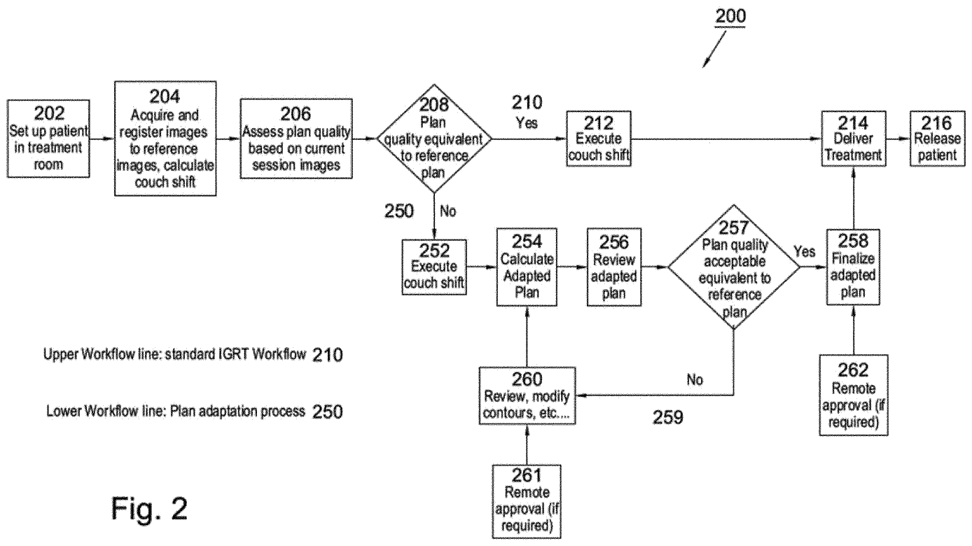

FIG. 2 shows a radiotherapy treatment workflow 200 according to the present disclosure. The radiotherapy treatment workflow 200, which may also be referred to as a radiotherapy workflow, begins at block 202, in which a patient is set up in a treatment room. The patient set up comprises positioning the patient on an adjustable patient support of a radiotherapy device, such as those discussed in relation to FIG. 1. The patient may be positioned for treatment in a particular fraction session of their prescribed and/or scheduled radiotherapy treatment plan (including but not limited to the first fraction session). In other words, the patient may be positioned at a particular position according to the scheduled radiotherapy treatment plan and the radiation delivery apparatus may be positioned and/or prepared to deliver a prescribed dose according to the scheduled radiotherapy treatment plan.

The radiotherapy workflow 200 continues to block 204, in which at least one image of the patient positioned on the adjustable patient support is obtained. The image may be obtained using known patient imaging techniques, such as x-ray imaging, CBCT imaging, or/and MR imaging. The at least one image of the patient is compared with a reference image associated with the scheduled radiotherapy treatment plan. The reference image may have been obtained or generated and/or selected during the initial planning stage, for example. The comparison between the images may be in accordance with known techniques for image registration. The reference image indicates a reference position for a region of interest (ROI) of the patient. The region of interest may comprise a position of the target/tumour, and associated dimensions, as well as other anatomical features of the patient, such as the positions and associated dimensions of nearby organs, and in particular organs at risk. In many situations, a comparison of the images may indicate a difference or discrepancy between the position of the ROI in the obtained image of the patient on the adjustable patient support and the reference position for the ROI in the reference image associated with the scheduled radiotherapy plan. The discrepancy may be caused by, for example, inter-fractional changes in patient anatomy, such as swelling, fluid retention or other anatomical changes due to, for example, movements or different filling levels of internal organs. Weight gain or loss during therapy may also lead to a gradually growing discrepancy. Based on the comparison, a positional adjustment of the adjustable patient support is determined, which may be referred to as a “couch shift” in examples in which a patient couch is used as the adjustable patient support. The positional adjustment would move a current position of the ROI to a second position in which the ROI is more closely aligned with the reference position for the ROI. Such an adjustment is desirable in order to improve the accuracy and/or precision of delivery of radiation to the target.

At block 206, the scheduled radiotherapy treatment plan is assessed by determining whether delivering the scheduled radiotherapy treatment plan with the ROI at the second position would meet at least one pre-defined treatment plan quality criterion. Pre-defined treatment plan quality criteria may include a minimum prescribed dose for the target, and/or a maximum prescribed dose for at least one nearby organ at risk (OAR) as calculated by the dose planning system using Monte Carlo or another suitable high quality algorithm. For example, implementing a positional adjustment to compensate for a discrepancy in the position of the target on the day of treatment compared to within the scheduled treatment plan may mean that delivering radiation according to that plan, e.g. with particular multi-leaf collimator position or arrangement, may deliver an unacceptably high dose to a nearby OAR, which is problematic, and so the scheduled treatment plan would be determined not to meet a quality criterion related to the maximum dose to be delivered to that OAR. Accordingly, the determination at the block 206 may comprise computing or evaluating geometrical and/or anatomical positions for the proposed delivery with the ROI at the second position, along with dose depth characteristics. At block 208 of the radiotherapy workflow 200, it is determined whether the proposed delivery of the scheduled treatment plan with the ROI at the second position is equivalent to the reference plan in terms of the at least one treatment plan quality criterion. At this point 208, advantageously, the radiotherapy workflow 200 can split into a first workflow path 210 or a second workflow path 250.

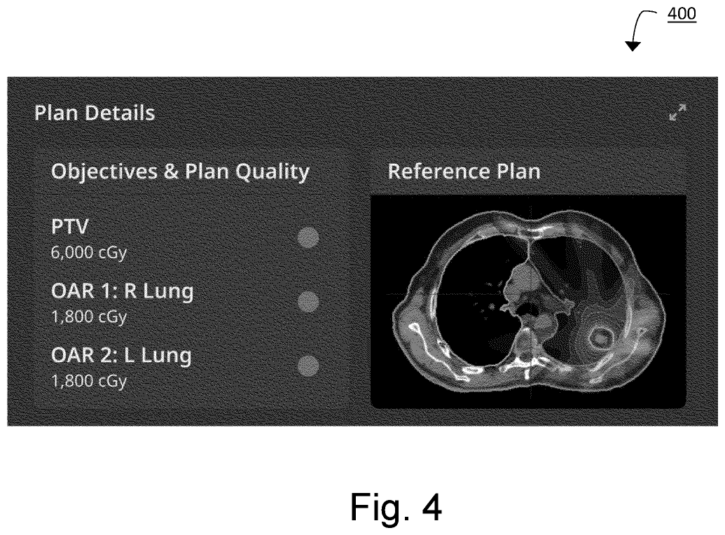

At block 208, the methods and systems disclosed herein involve displaying a visual indicator of whether delivering the scheduled radiotherapy treatment plan with the ROI at the second position would meet the at least one treatment plan quality criterion, and enabling selection, by a user, of one of the first workflow path 210 or the second workflow path 250. A suitable visual indicator is depicted in FIGS. 4 to 7 and described in the accompanying parts of the description.

The first workflow path 210 comprises executing 212 the positional adjustment to move the ROI to the second position, and delivering 214 treatment according to the originally scheduled radiotherapy treatment plan. The patient is then released at block 216, after which the workflow ends. Thus, the first workflow path 210 corresponds to conventional IGRT in which a treatment plan is developed in advance of treatment and then delivered without online adaptation.

The second workflow path 250 also comprises executing 252 the positional adjustment to move the ROI to the second position. At the next block 254, an adapted radiotherapy plan to be delivered with the ROI at the second position is calculated. Such a calculation may be performed by determining new radiation delivery parameters, such as beam shaping parameters (which may relate to a collimator component, which may be a multi-leaf collimator), that would deliver the prescribed dose for this fraction to the target while meeting the prescribed clinical objectives. For example, the new radiation delivery parameters may allow the prescribed dose to be delivered without exceeding the maximum dose that can be delivered to a nearby OAR according to the treatment plan quality criterion. Calculating a new radiotherapy treatment or delivery plan can take considerable time, and may typically require optimisation of a multitude of variables, such as beam energy, dose rate, beam directions, speed of movement and modulation of fluence, and include review by a clinician, and it is conventionally not the preferred approach to calculate a new plan with the patient in-situ. However, the present approach reduces the complexity of calculating a new plan by enacting the positional adjustment to place the ROI at the second position in which, by earlier comparison with the reference image, it is closer to the reference position. Accordingly, the optimization problem, and thus computation time is reduced. Furthermore, the resulting adapted radiotherapy plan may then be evaluated by using the same fewer plan evaluation criteria (or pre-defined treatment plan quality criterion/criteria) that have been established as being the most important criteria/criterion for the reference plan, rather than a full plan evaluation of all OARs or criteria. Using simplified plan criteria for the evaluation and comparing those to the reference plan, rather than performing a full plan quality analysis as would be done for a completely new plan provides the confidence that the adapted radiotherapy plan meets those constraints that are considered most important. Accordingly, the adapted radiotherapy plan is calculated based on a subset of the parameters/variables that were used to calculate the scheduled radiotherapy treatment plan, rather than the full set of variables, reducing computational burden. For example, the adapted radiotherapy plan may be calculated based on parameters corresponding to the at least one pre-defined treatment plan quality criterion, and in some examples, based only on parameters corresponding to the at least one pre-defined treatment plan quality criterion. In particular examples, the adapted radiotherapy plan may be calculated based only on parameters corresponding to one or more pre-defined treatment plan quality criteria that would not be met by the scheduled radiotherapy treatment plan with the ROI at the second position. In general, the present approach allows the workflow to switch from conventional IGRT to online adaptive radiotherapy on a fraction-to-fraction basis without excessively extending the amount of time the patient is held in position for treatment.

Although not shown in FIG. 2, in some examples, following the calculating 254, the adapted radiotherapy plan is then delivered to the patient and the patient is subsequently released, ending the workflow. However, optionally, the second workflow may comprise a further block 256 of reviewing the adapted plan in a manner like that of blocks 206 and 208 in order to double-check the plan. A determination is made at block 257 as to whether the adapted radiotherapy plan is acceptable or equivalent to the reference scheduled radiotherapy treatment plan. If the adapted radiotherapy plan is found to be acceptable or equivalent to the reference plan in terms of the at least one pre-defined treatment plan criterion, the plan is finalised 258, before being delivered 214. Optionally, a remote approval procedure 259 allows an experienced clinician to approve the finalised plan without needing to be on site. If the adapted radiotherapy plan is found not acceptable or not equivalent to the reference plan in terms of the at least one pre-defined treatment plan criterion, then a review 260 is performed, which may comprise a close review and potentially modifying contours in order to achieve a better result after a further re-optimization, and/or may involve providing the plan for remote review 261, such as by an experienced clinician, in order to assess any potential compromises if the plan quality criteria cannot be fully met. The new adapted radiotherapy plan may then be assessed against the at least one treatment plan quality criterion as in the prior assessment block 256. In some examples, an adapted radiotherapy plan that already does meet the at least one treatment quality criterion is provided for remote review, such as by an experienced clinician. Likewise, an optional remote approval step to finalise the adapted plan may be provided at block 262, if required. “Remote review” means that the review process is performed off-site, and not at the site of the radiotherapy treatment session.

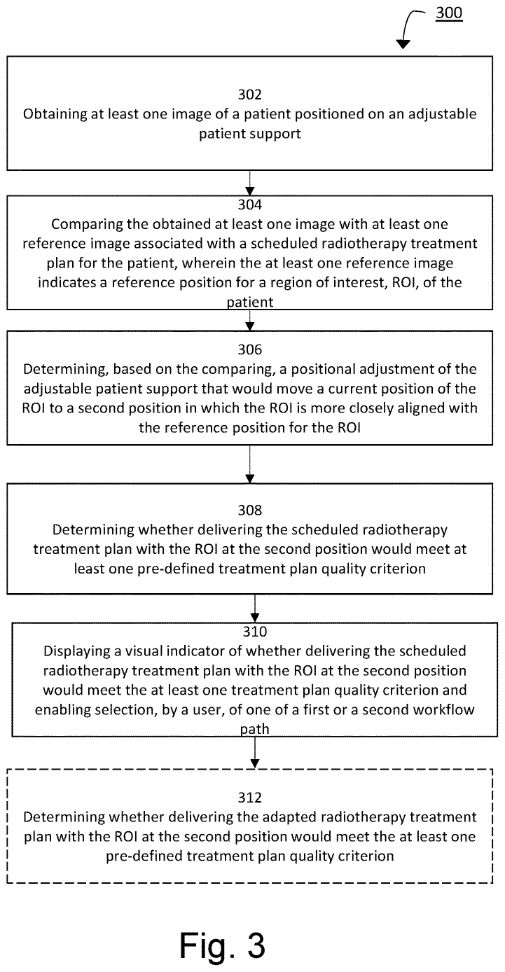

FIG. 3 shows a computer-implemented method 300. The method is suitable for being performed on a radiotherapy treatment day, when a patient is to have a fraction of radiation delivered to a region of interest according to their scheduled treatment plan. The radiotherapy workflow 200 of FIG. 2 is an exemplary implementation of method 300. Each of the blocks of the method 300 may be performed in accordance with a corresponding block(s) of the radiotherapy workflow 200 of FIG. 2, including optional exemplary implementations associated with a particular block. However, the method 300 is more general, and need not comprise all of the stages of the radiotherapy workflow 200 of FIG. 2.

The method 300 comprises, at block 302, obtaining at least one image of a patient positioned on an adjustable patient support. As described above in relation to FIG. 2, this image may be an MR image, a CT, CBCT image, or another medical imaging modality. At block 304, the method 300 comprises comparing the obtained at least one image with at least one reference image associated with a scheduled radiotherapy treatment plan for the patient, wherein the at least one reference image indicates a reference position for a region of interest, ROI, of the patient. The at least one reference image may be an image using during generation of the patient's radiotherapy treatment plan, for example. As described above, comparing may comprise registering the at least one image obtained at block 302 with the at least one reference image obtained at block 304.

Alternatively, comparing the images may comprise determining a degree of similarity between the patient anatomy in the images. The degree of similarity may be, for example, a dice similarity coefficient or the Jaccard index. The degree of similarity may be determined only for a tumour, OAR, or other anatomical region of interest, in which case auto-contouring tools may be used to isolate the structure(s) of interest before the calculation of the degree of similarity is performed.

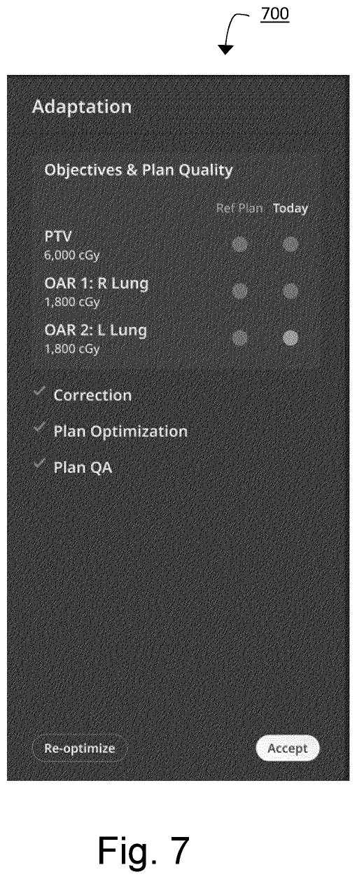

At block 306, the method 300 comprises determining, based on the comparing, a positional adjustment of the adjustable patient support that would move a current position of the ROI to a second position in which the ROI is more closely aligned with the reference position for the ROI. For example, based on the registered images, it may be determined that there is a discrepancy or ‘offset’ between the position of an ROI in the reference image(s) and the position of the ROI in the newly obtained image(s). It may further be determined that this offset can be ‘corrected for’, or minimised, by re-positioning the patient using a positional adjustment to be enacted via the patent support surface. Example positional adjustments are shown in FIGS. 4 and 7 and described in the accompanying description paragraphs below.

At block 308, the method comprises determining whether delivering the scheduled radiotherapy treatment plan with the ROI at the second position would meet at least one pre-defined treatment plan quality criterion. In an example, the pre-defined treatment plan quality criterion is the prescribed dose to be delivered to the target during that fraction of treatment. In some examples, multiple pre-defined treatment plan quality criteria are used, which may include the prescribed dose to be delivered to the target (also known as the planning target volume, PTV, dose) as well as a maximum allowable dose for one or more nearby organs at risk. Further examples are provided below in relation to FIGS. 4 to 7.

In another example, the pre-defined treatment plan quality criterion is a threshold degree of similarity between the patient anatomical structure, when comparing the at least one reference image with the at least one obtained image (i.e. with the at least one session image). If the images have a similarity score above the threshold, then treatment may continue according to the original plan and no adaptive re-planning may be required. In this scenario, the first workflow path may be followed and/or recommended to the clinician. If the degree of similarly is below the threshold, then adaptive re-planning may be required. In this scenario, the second (adaptive) workflow path may be followed and/or recommended to the clinician.

In an implementation, two different treatment plan quality criteria are used. For example, a dose-related criterion and a similarity-related criterion. If the similarity score is above the first similarity threshold, then the plan continues, or is recommended to continue, as planned. If the similarity score is below the first but above the second similarity threshold, then it is determined whether a dosimetric criteria/threshold is met, in the manner described elsewhere herein. If the dosimetric criteria is nonetheless met even though the first similarity threshold is not met, then the treatment may continue, or is recommended to continue, as planned. Finally, if the second (lower) similarity threshold is not met, then the plan is recommended for adaptation.

At block 310, the method comprises displaying a visual indicator of whether delivering the scheduled radiotherapy treatment plan with the ROI at the second position would meet the at least one treatment plan quality criterion and enabling selection, by a user, of one of a first or a second workflow path, wherein the first workflow path comprises executing the positional adjustment and delivering treatment according to the scheduled radiotherapy treatment plan, and wherein the second workflow path comprises executing the positional adjustment and calculating an adapted radiotherapy plan that, when delivered with the ROI at the second position, would meet the at least one treatment plan quality criterion. The visual indicator is chosen to provide an advantage that it is very quick and easy for a user to interpret, and reduces the cognitive load required for a user to interpret a complex situation. For example, the visual indicator may comprise a coloured icon. The visual indicator may be one of a range of possible visual indicators, each comprising a colours, in which a first colour indicates that delivering the scheduled radiotherapy treatment plan with the ROI at the second position would meet the at least one pre-defined treatment plan quality criterion and a second colour indicates that delivering the scheduled radiotherapy treatment plan with the ROI at the second position would not meet the at least one pre-defined treatment plan quality criterion. In examples in which more than one pre-defined treatment plan quality criterion is used, the visual indicator may comprise a respective colour, or coloured icon, for each criterion.

The colour may correspond to whether or not the scheduled (or adapted) treatment plan meets the at least one pre-defined treatment plan quality criterion by, for example, showing green if it does, or red if it does not. In some examples, a yellow or amber colour may be displayed, showing, for example, that the scheduled treatment plan being delivered with the ROI at the second position may not correspond to the original quality desired, but is of acceptable quality. Advantageously, use of colours, such as in a “traffic light” arrangement of green, red, amber, is allowed within regulations for the design of radiotherapy systems and is a widely understood convention. Furthermore, the use of colour lightens a cognitive burden on clinicians and renders what was previously a large number of complex factors into a single, simple indicator. Examples of the visual indicator are provided in FIGS. 4 to 7 and within the accompanying description below.

In some examples, displaying the visual indicator may comprise displaying, for each of a plurality of treatment plan quality criteria, a respective visual indicator of whether delivering the scheduled radiotherapy treatment plan with the ROI at the second position would meet the respective treatment plan quality criterion. For example, for each of the plurality of treatment plan quality criteria, a respective red or green colour is indicated.

The method 300 optionally further comprises an additional block 312 of determining, within the second workflow path 250, whether delivering the adapted radiotherapy treatment plan with the ROI at the second position would meet the at least one pre-defined treatment plan quality criterion.

The method 300 may yet further comprise displaying a visual indicator of whether delivering the adapted radiotherapy treatment plan with the ROI at the second position would meet the at least one treatment plan quality criterion, and enabling selection, by a user, of one of delivering treatment according to the adapted radiotherapy plan; or calculating a further adapted radiotherapy treatment plan, such as in block 260.

As set out above, the at least one treatment plan quality criterion comprises one or more of a minimum target dose threshold and a maximum organ at risk dose threshold. In some examples, respective maximum organ at risk dose thresholds are used for multiple organs. In some examples, fewer parameters or variables are used for the treatment plan quality criterion than were used in developing the initial treatment plan, allowing the assessment of the plan to be performed quickly and efficiently. In some examples, calculating the adapted radiotherapy plan comprises calculating multi-leaf collimator component parameters to be used in delivering treatment, and/or other radiotherapy delivery variables, such as gantry parameters.

Each of the implementations of the method 300 may be performed in a radiotherapy fraction session, i.e. in a single session with a patient positioned on the adjustable patient support.

Several example implementations of FIG. 3 will now be described with reference to FIGS. 4 to 7. FIGS. 4 to 7 relate to an exemplary patient with associated treatment plan and clinical goal. In the illustrative scenario described below, a scheduled treatment plan has been developed for the patient, with pre-defined treatment plan quality criteria relating to a target dose and OAR maximum doses, and FIGS. 4 to 7 relate particularly to exemplary implementations of blocks 308 and 310 of the method 300 of FIG. 3.

FIG. 4 shows an example portion 400 of a radiotherapy user interface according to the present disclosure. The portion 400 of the user interface shows scheduled radiotherapy plan details for this exemplary scenario. On the left-hand side, “objectives” are indicated, corresponding to a plurality of pre-defined treatment plan quality criteria, including a planning target volume (PTV) dose of at least 6.00 Gy, and two OAR maximum doses of 1.8 Gy each for the right (R) lung and left (L) lung. It will be understood that in other examples, other values and pre-defined treatment plan quality criteria may be used. Alongside each objective is a colour icon indicator that indicates whether or not the scheduled treatment plan satisfies that criterion. On the right-hand side of the portion 400 of FIG. 4 is an image of the reference plan showing the ROI of the patient, comprising the target and two lungs. Again, it will be understood that for other targets and/or ROIs, other images will be shown.

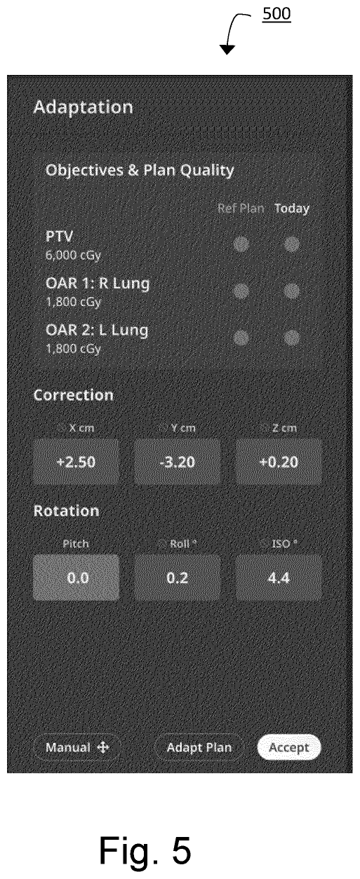

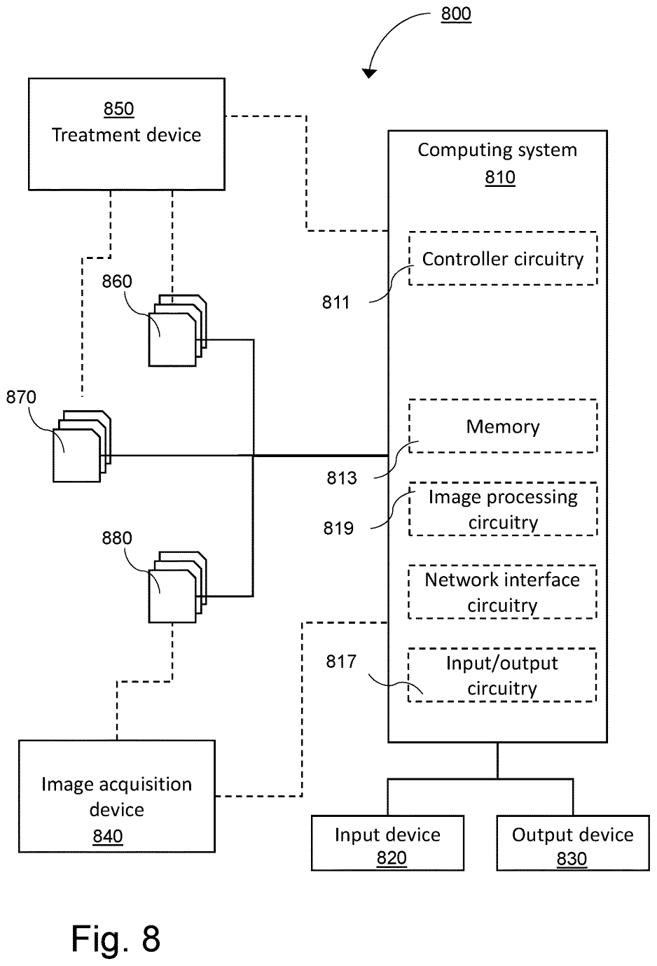

FIGS. 5-7 show further example portions 500, 600, 700 of an exemplary radiotherapy user interface according to the present disclosure. The user interface may be implemented using a display screen, such as a touch screen, or other screen. Further examples of input and/or output devices for the user interface are provided herein in relation to FIGS. 1 and 8.

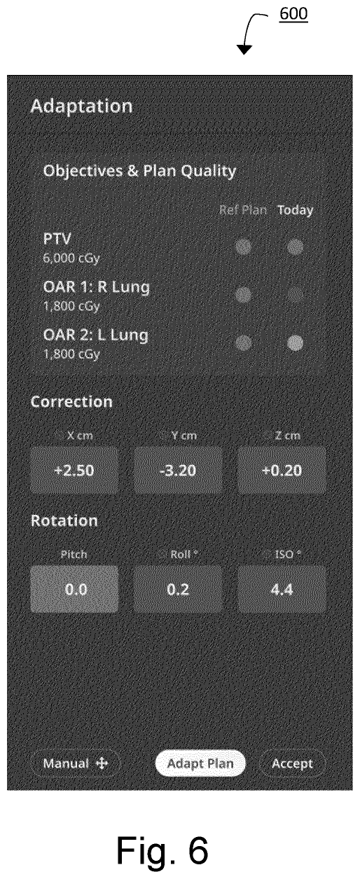

The user interface enables a user to select either the first or second workflow paths in accordance with the methods and systems disclosed herein. In FIG. 5, exemplary objectives/quality criteria and plan quality visual indicators are shown. A first, left-hand column shows a visual indicator for the reference plan, and a second, right-hand column shows a visual indicator for “Today”, which is based upon the assessment made based on the comparison of the patient image obtained during the session with the reference image of FIG. 4, and the determined positional adjustment to be made to the ROI, as in the method 300 of FIG. 3. Shown below that are “Correction” and “Rotation” sections that display exemplary determined positional adjustments (as determined in block 306) in terms of X, Y, Z coordinates and pitch, roll, and yaw angles of the adjustable patient support. The user is presented with buttons corresponding to “Adapt Plan” or “Accept”, which in this example are near the bottom of the user interface but need not be located so. The user is thereby enabled to select the first workflow path or the second workflow path as in block 310. Since in the example portion of FIG. 5, the scheduled treatment plan meets all of the pre-defined treatment plan quality criteria, the “Accept” button is highlighted as an additional visual indicator, prompting the user to select the first workflow path 210 and proceed with conventional treatment. In the example of FIG. 6, on the other hand, the OAR 1: R Lung criterion is not met if implementing the determined positional adjustment. Accordingly, the “Adapt Plan” button is highlighted to the user as a visual indicator, and the user will select the “Adapt Plan” button to launch the second workflow path and adapt the plan in accordance with the methods described herein. Subsequently, an adapted plan will be calculated, and the user may be presented with a user interface like that of FIG. 7. The visual indicators shown in FIG. 7 show that the “Today” plan, which has just been calculated, meets the pre-defined treatment quality criteria for PTV and OAR 1: R Lung. The criterion for OAR 2: L Lung is shown as yellow or amber, which may indicate that the parameter value provided by the adapted plan is not ideal but is acceptable. Accordingly, the user may use the highlighted “Accept” button to accept and implement the adapted plan or may choose to “Re-optimize” and calculate a further adapted plan, as in the optional blocks of the second workflow path 250.

In each example, the user can immediately see the visual indicator(s) provided and so the cognitive burden for the user is lowered. Further, the computational burden of adapting the radiotherapy plan can be lowered by the approaches disclosed herein, such as by executing the positional adjustment in order to remove the need to re-calculate patient positioning and/or re-obtain a reference image. Accordingly, the time that a patient is held in a potentially uncomfortable position is reduced.

Each of FIGS. 4 to 7 may be used in the method 300 of FIG. 3 and/or radiotherapy workflow 200 of FIG. 2.

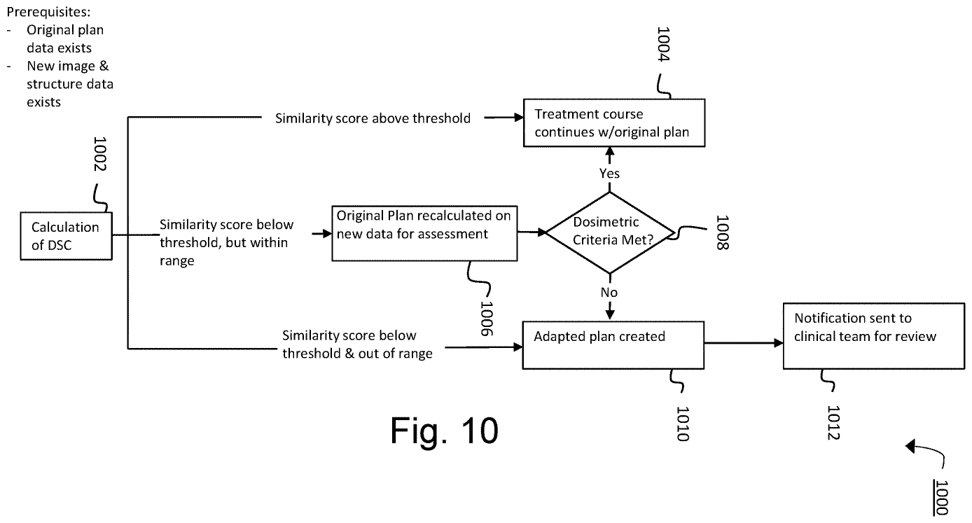

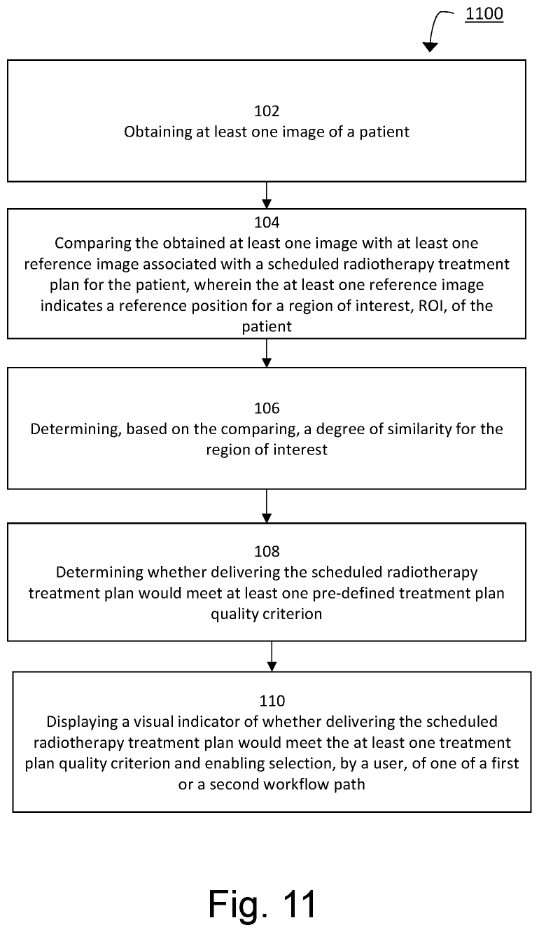

Reference is made herein to determining a treatment plan quality criterion based on a degree of similarity between anatomical structure in an original image, and the same anatomical structure in an image obtained on the day of treatment. The flowcharts of FIGS. 10 and 11 describe example functionality which makes use of the degree of similarity.

At block 1002, the degree of similarity is determined. In an example, the degree of similarity is the dice similarity coefficient (DSC). If the anatomical structures are sufficiently similar, i.e. if the DSC is above a first threshold, treatment may continue at block 1004 as planned, e.g. according to a first workflow path.

If the DSC is below the first threshold, but above a second (lower) threshold, then the dose which would be received by the anatomical structure (region of interest) according to the current treatment plan is determined using known methods such as Monte Carlo methods. The original plan may be recalculated using the new patient image, and a dose value determined for the region of interest. If the dose value meets a dosimetric criteria at block 1008, then the treatment may continue in accordance with the original plan at block 1004. If however the dosimetric criteria is not met, then the method proceeds to block 1010. At block 1010, an indication is displayed to the clinician that the plan should be adapted. This indication may take the form described above with respect to block 30 and FIGS. 4 to 7 to enable selection of the appropriate workflow path by the clinician. Alternatively, the adapted plan may be automatically created. After creation of the adapted plan, an indication/notification may be output to the clinician for review at block 1012.

In a particular implementation, the method may use the dice similarity coefficient (DSC) to automatically quantify the similarity between the same anatomical structure (target or organ at risk) on original vs new images and automatically, or semi-automatically, trigger the most appropriate next-action based on a user-defined threshold value. This process may involve manual preparation tasks in regard to import and contouring of new data or could be performed automatically in the system backend using advanced deformable structure propagation or auto-contouring tools to prepare data.

Based on a clinical protocol, the most important structures may be identified and if their similarity score is above the threshold, no corrective or adaptive action is required and the treatment course can continue as planned. If a similarity score is below the threshold, but still in an appropriate range, an assessment workflow of the original plan on the new data can be automatically triggered within the system to validate the appropriateness. The system will then report the outcome of the assessment to the clinical team if no significant differences are observed or trigger an adaptive re-plan if significant dosimetric differences are observed, with the clinical team then notified to review the output of both the original plan assessment and the adapted plan. If a similarity score is well below the threshold then an adapted plan activity is automatically triggered, with a notification to the clinical team that the original plan is no longer appropriate and that an adapted plan is ready for review.

This automated assessment tool allows for simplification of the decision making process and removes elements of subjectivity in choosing the next action for the patient.
1. Reduced clinician overhead (and costs associated) as the structure similarity review task is automated, with a next-action then triggered based on the outcome of this task, removing subjectivity or pressured assessments conducted by an unqualified staff member
2. Improved patient outcomes through the automation of many typically manual processes, with greater opportunity for employing adaptive plan assessment and adaptive replanning at a greater frequency
3. Reduction in variability of adaptive workflows may allow for clinics to quantify when these tasks are performed to insurance/reimbursement bodies, creating opportunities for financial benefit to a facility.

Making use of a similarity metric can be incorporated into the methods and systems of the present disclosure in any appropriate way as would be understood by the skilled person. The degree of similarity may form the basis, for example, of the treatment plan quality criterion at block 308, for example.

FIG. 11 is a flowchart depicting a method 1100 according to the present application. Blocks 102 and 104 may be equivalent to blocks 302 and 304. At block 106, a degree of similarity is determined. At block 108, it is determined whether delivering the scheduled radiotherapy treatment plan with the ROI at the second position would meet at least one pre-defined treatment plan quality criterion, where the criterion is based on the degree of similarity determined at block 106. This block may incorporate the similarity thresholds as detailed above with respect to FIG. 10, for example.

Finally, block 110 comprises displaying a visual indicator of whether delivering the scheduled radiotherapy treatment plan would meet the at least one treatment plan quality criterion and enabling selection, by a user, of one of a first or a second workflow path. This block may be equivalent to block 310 of FIG. 3. In an example, if a first similarity threshold is met, then a green visual indicator may be displayed, enabling selection of the first workflow path. If the first similarity threshold is not met, then a n amber or red visual indicator may be displayed, enabling selection of the second workflow path and adaptation of the plan.

As discussed above, currently available online adaptive treatment workflows require a lot of manual steps and decisions, each of which require appropriate skills and clinical experience at the point of treatment delivery. In other words, current online adaptive workflows require a high level of expertise and experience to use effectively, and experienced clinicians must be present on-site for the workflow to operate, which is time and resource intensive. Often, the patient is in a position for treatment during the time duration required for calculating the adapted treatment plan. In some implementations, therefore, it may be desirable to increase the speed at which a treatment plan may be changed, modified, adapted and/or replaced, thereby enabling improved patient experience, improved patient throughput, as well as reducing the time and resource requirements for clinicians.

Furthermore, since the adapted treatment plan is often generated based on an image generated with the patient in the treatment position, it is desirable that the patient does not move between generation of the image and application of the resulting daily treatment plan. However, since generation of a treatment plan is computationally complex, it can also be time consuming. For example, it may be 5-20 minutes after the image is taken that the adapted treatment plan is ready for delivery. Over this timescale, the patient may experience discomfort and may, potentially inadvertently, move relative to the position they were in when the image was generated, and may be more likely to move during the treatment itself. This may result in the adapted treatment plan being less applicable to the new position of the patient. The more the position and internal anatomy of the patient differ relative to the reference image on which the initially-scheduled treatment plan is based, the more substantial and more time-consuming may be the adaptation required to generate the adapted treatment plan.

Moreover, previous approaches to adaptive treatment planning may not be as flexible as would be desired. For example, previous approaches may simply adapt the most recently generated treatment plan, which may be suboptimal. In addition, previous approaches may be limited in the imaging modalities which can be used as the basis for generating treatment plans, which may not be optimal for tumours in different parts of subject anatomies. In addition, it should be appreciated that some of the delay between an image being taken and treatment commencing, as described above, may be due to a clinician evaluating various characteristics of a treatment plan and navigating a workflow, which may take longer than would be desirable according to previous approaches.

Accordingly, there is a need for more efficient generation of radiotherapy treatment plans. There is also a need for reducing the complexity involved in generation of radiotherapy treatment plans. There is also a need for more flexibility and/or more versatility in the generation of radiotherapy treatment plans. It would also be advantageous to increase the accuracy and/or the efficiency of radiotherapy treatments.

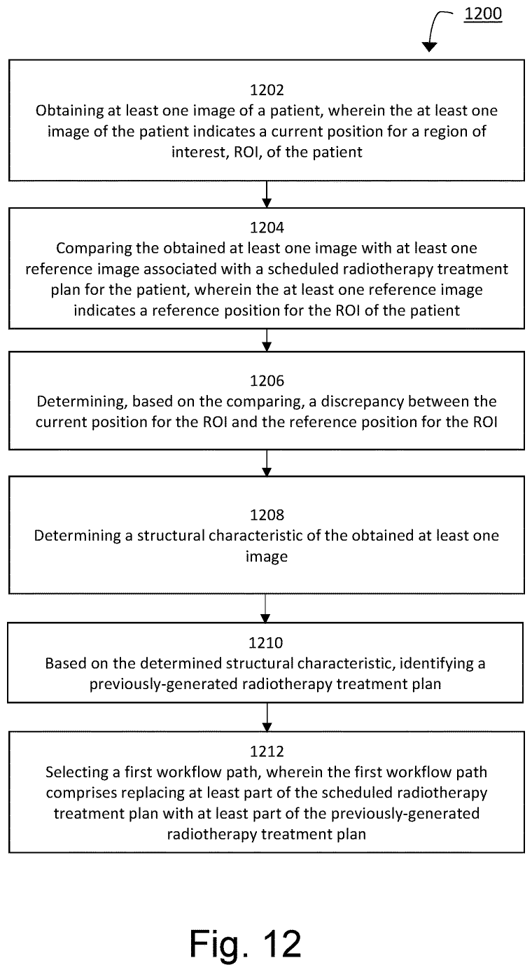

FIG. 12 shows a method 1200 for use in a radiotherapy workflow. The method 1200 may be a computer-implemented method. The method 1200 is suitable for being performed on a radiotherapy treatment day, when a patient is to have a fraction of radiation delivered to a region of interest according to their scheduled treatment plan.

At a first block 1202, the method 1200 comprises obtaining at least one image of a patient, wherein the at least one image of the patient indicates a current position for a region of interest, ROI, of the patient. The patient may be positioned for treatment in a particular fraction session of their prescribed and/or scheduled radiotherapy treatment plan (including but not limited to the first fraction session). In other words, the patient may be positioned at a particular position according to the scheduled radiotherapy treatment plan and the radiation delivery apparatus may be positioned and/or prepared to deliver a prescribed dose according to the scheduled radiotherapy treatment plan. The obtained image may be an MR image, a CT, CBCT image, or another medical imaging modality.

At a block 1204, the method 1200 comprises comparing the obtained at least one image with at least one reference image associated with a scheduled radiotherapy treatment plan for the patient, wherein the at least one reference image indicates a reference position for the ROI of the patient. Blocks 1202 and 1204 may be equivalent to blocks 302 and 304 of FIG. 3 and/or blocks 102 and 104 of FIG. 11 (and vice versa).

At block 1206, the method 1200 further comprises determining, based on the comparing at block 1204, a discrepancy between the current position for the ROI and the reference position for the ROI.

The discrepancy may indicate that it would be preferable to adapt and/or modify the scheduled radiotherapy treatment plan. For example, it may be determined that, based on the discrepancy between the current position for the ROI and the reference position for the ROI, delivering the scheduled radiotherapy treatment plan with the patient at the current position would not meet at least one pre-defined treatment plan quality criterion.

The discrepancy may comprise a positional and/or shape discrepancy. In some examples, the discrepancy comprises at least one of: a difference in position for the ROI, a difference in location for the ROI, and/or a difference in shape for the ROI. For example, the discrepancy may correspond to a difference in position of at least one point and/or part of the ROI when comparing the obtained at least one image with the at least one reference image associated with the scheduled radiotherapy treatment plan for the patient. For example, the ROI may have moved within the patient to another position and/or location since the reference image was obtained. Such a discrepancy may be addressed by taking an “adapt to position” approach such as by implementing a shift of the patient on an adjustable patient support. An adapt to position approach may involve a rigid registration of the two images and recalculation of the treatment plan to improve the target coverage at the new position. Additionally or alternatively, the discrepancy may be a shape discrepancy, such that there is a difference in shape of the ROI between the ROI of the obtained at least one image and the ROI of the reference image. Such a discrepancy may be addressed by taking an “adapt to shape” approach, which may involve deformable image registration. A difference, or discrepancy, in shape may be caused by, for example, a change in the internal organs of the patient, such as a change in bladder filling for the present situation compared with the situation in which the reference image was obtained.

At block 1208, the method 1200 further comprises determining a structural characteristic of the obtained at least one image.

The structural characteristic may be based on segmentation and/or statistical analysis of the image. In some examples, the determined structural characteristic is representative of at least one of: a region of interest, a treatment target, and/or an organ-at-risk, a Hausdorff distance, a volume, and/or an overlap volume histogram. For example, a Hausdorff distance may represent a maximum distance between structures in the image. An overlap volume histogram may be used to define an amount of overlap between structures, such as an overlap of a target and organ-at-risk. The structural characteristic may be representative of a volume of a treatment target, region of interest, and/or organ-at-risk.

In some examples, the determined structural characteristic is representative of a plurality of anatomical structures, and/or may represent a structural and/or statistical metric or score for the image. The structural and/or statistical metric or score may represent the image as a whole or may represent a portion of the image, such as any one or more of the structures and/or volumes described herein. In some examples, the structural characteristic may be used in making a similarity comparison with another image, and/or may correspond to a Dice similarity coefficient (DSC). The method 1200 may comprise using the Dice similarity coefficient to automatically quantify the similarity between the same anatomical structure (target, region of interest, and/or organ at risk) in the obtained image and in another image, such as the reference image and/or an image associated with a previously-generated radiotherapy treatment plan. Additionally or alternatively, other measures or tools for determining the similarity of two images may be used, such as the Jaccard index, or such as a comparison between one or more of a Hausdorff distance, volume of a target, and/or overlap volume histogram. In some examples, based on a clinical protocol, the most important structures may be identified in one or more of the obtained image and the other image and a structural characteristic of the respective image may be based on those.

At block 1210, the method 1200 further comprises, based on the determined structural characteristic, identifying a previously-generated radiotherapy treatment plan.

In some examples, identifying the previously-generated radiotherapy treatment plan comprises comparing the determined structural characteristic of the obtained at least one image with a structural characteristic associated with the previously-generated radiotherapy treatment plan. In some examples, identifying the previously-generated radiotherapy treatment plan further comprises determining a similarity between the determined structural characteristic of the obtained at least one image and a structural characteristic associated with the previously-generated radiotherapy treatment plan. The similarity may be determined as a similarity score, and/or a similarity metric, and/or a degree of similarity, including any of the similarity or comparison metrics and structural characteristics described herein. In some examples, identifying the previously-generated radiotherapy treatment plan further comprises determining a Dice similarity coefficient for the obtained at least one image and an image associated with the previously-generated radiotherapy treatment plan. The similarity may be determined only for a tumour, OAR, or other anatomical region of interest, in which case auto-contouring tools may be used to isolate the structure(s) of interest before the calculation of the degree of similarity is performed.

For example, the previously-generated radiotherapy treatment plan may be associated with a previous image used to generate and/or calculate that plan, and that previous image may have its own structural characteristic(s). In such examples, the previously-generated radiotherapy treatment plan may be identified based on the determined structural characteristic by an assessment or comparison between the structural characteristic of the image obtained at block 1202 and that of the previous image associated with the previously-generated radiotherapy treatment plan. Such an assessment or comparison may take the form of the similarity comparison examples set out above, such as the calculation of a DSC and/or Jaccard index, and/or a comparison of a Hausdorff distance in the obtained at least one image with a Hausdorff distance in the previous image associated with the previously-generated radiotherapy treatment plan, for example. In each example, the previously-generated radiotherapy treatment plan may be identified and/or selected based on the similarity score, metric, and/or degree exceeding a pre-defined threshold.

The previously-generated radiotherapy treatment plan may be suitable for delivering the prescribed dose to the patient, and in particular in relation to present arrangement of the ROI. The previously-generated radiotherapy treatment plan may be determined to be suitable on the basis of the structural characteristic and/or structural similarity comparison. As described herein in relation to other radiotherapy treatment plans in general, the previously-generated radiotherapy treatment plan may comprise a treatment schedule component, e.g. how the dose is divided into fractions and when those fractions are delivered, and a radiotherapy delivery plan, which determines particular system variables or parameters for delivering a particular dose fraction to a particular site, such as suitable beam shaping component positions or configurations, beam angles, beam energies, or/and duration of exposure. The previously-generated radiotherapy treatment plan may comprise beam segments.

As described above, the previously-generated radiotherapy treatment plan may be associated with an image originally used to calculate the previously-generated radiotherapy treatment plan, and that image may in turn have an associated structural characteristic. The previously-generated radiotherapy treatment plan may be associated with a similar and/or identical type of structural characteristic as that determined for the obtained image and may be identified and/or selected on that basis. For example, a comparison may be made between the structural characteristic(s) of the obtained image and a structural characteristic of an image used to generate the previously-generated radiotherapy treatment plan. If the images show sufficient similarity in terms of their structural characteristic(s), the previously-generated radiotherapy treatment plan may be considered suitable for delivering the scheduled prescribed dose to the patient in relation to the current state of the patient. Accordingly, the previously-generated radiotherapy treatment plan may deliver the prescribed dose more effectively than the scheduled radiotherapy treatment plan when taking into account the discrepancy determined at block 1206.

The previously-generated radiotherapy treatment plan may be associated with the same patient as the patient for which the obtained image is obtained at block 1202, or may be associated with another patient. The other patient may be part of a same patient cohort as the patient for which the obtained image is obtained. For example, all data from a particular treatment cohort may be stored and plans previously created for different patients could be reused for another patient if their structures share similarity, given that prescription and protocol parameters shall typically be standardised.

At block 1212, the method 1200 further selecting a first workflow path, wherein the first workflow path comprises replacing at least part of the scheduled radiotherapy treatment plan with at least part of the previously-generated radiotherapy treatment plan.

In some examples, the selecting may be performed based on determining that the previously-generated radiotherapy treatment plan is preferable to the scheduled radiotherapy treatment plan for delivering the prescribed dose given the discrepancy determined at block 1206. For example, it may be determined that the previously-generated radiotherapy treatment plan meets at least one pre-defined treatment plan quality criterion.

The first workflow path enables quick modification, adaptation, changing, and/or replacement of the scheduled radiotherapy treatment plan to take into account the discrepancy without requiring full calculation of an adapted plan. By replacing, at least in part, the scheduled radiotherapy treatment plan with the previously-generated radiotherapy treatment plan, the prescribed dose may be more effectively delivered to the patient in their current state. Accordingly, the scheduled radiotherapy treatment plan may effectively be “adapted” without incurring the time burden of fully calculating and/or verifying an adapted radiotherapy treatment plan. The approaches disclosed herein allow for a stored previously-generated radiotherapy treatment plan to be matched to a patient's current anatomical state based on historical structure data associated with the stored plan. The approaches disclosed herein may be particularly beneficial for cancers in the pelvic region, where anatomical variance is observed daily based on a patient's consumption of solids and liquids. Using such approaches enables a decreased burden on the clinical team to create a ‘new’ treatment plan if a patient requires adaptive radiotherapy. Furthermore, the approaches disclosed herein can provide improved patient outcomes through the automation of many typically manual processes, with greater opportunity for employing adaptive practices at a greater frequency. Yet furthermore, the approaches herein can reduce the need to rely on significant computational resources to create adapted plans and can provide a quicker adaptive process, minimizing total treatment time and allowing a clinic higher patient throughput.

The scheduled radiotherapy treatment plan may be replaced at least in part by modifying at least one of the plan parameters as disclosed elsewhere herein, such as multi-leaf collimator parameters, and/or by modifying the beam segments to be delivered. In some examples, the prescribed dose to the target could be modified. In some examples, the scheduled radiotherapy treatment plan may be replaced entirely by the previously-generated radiotherapy treatment plan.

The method 1200 may further comprise performing the steps of the first workflow path, but need not necessarily comprise those steps in all examples. In some examples, the first workflow path comprises delivering treatment to the patient according to the previously-generated radiotherapy treatment plan.

In some examples, identifying the previously-generated radiotherapy treatment plan comprises retrieving the previously-generated radiotherapy treatment plan from a repository comprising a plurality of previously-generated radiotherapy treatment plans. The repository may be a reference library or other storage comprising a plurality of previously-generated radiotherapy treatment plans, which may each respectively be previously-adapted radiotherapy treatment plans or non-adapted radiotherapy treatment plans. Each stored plan may be associated with a respective corresponding stored image, the image having been used to generate that plan, and/or a respective stored structural characteristic.

In some examples, the previously-generated radiotherapy treatment plan may be selected from a plurality of candidate previously-generated radiotherapy treatment plans based on having an associated structural characteristic that is most similar to the structural characteristic of the obtained image of the patient.

In some examples, the method 1200 further comprises the first workflow path further comprises determining that the identified previously-generated radiotherapy treatment plan is suitable for delivering a prescribed dose associated with the scheduled radiotherapy treatment plan.

In some examples, the method 1200 further comprises determining that the previously-generated radiotherapy treatment plan meets at least one pre-defined treatment plan quality criterion, the at least one pre-defined treatment plan quality criterion comprising one or more of: a minimum target dose threshold, a maximum organ at risk dose threshold, and the ROI exhibiting a threshold degree of similarity between the at least one obtained image and the at least one reference image.

In some examples, the method 1200 further comprises displaying information representative of dose characteristics associated with the identified previously-generated radiotherapy plan. For example, dosimetric goals, 3D dosimetry, and dose-volume histogram (DVH) information may be displayed to allow a clinician to verify that the selected previously-generated radiotherapy treatment plan is appropriate for use during the patient's session. If it is inappropriate, a user may perform a plan adaptation using an adaptive planning workflow, including those described herein. The display may be part of a user interface similar to or in accordance with the example portions 500, 600, 700 of an exemplary radiotherapy user interface described above. The user interface may be implemented using a display screen, such as a touch screen, or other screen. Further examples of input and/or output devices for the user interface are provided herein in relation to FIGS. 1 and 8. The user interface may enable a user to select a workflow path in accordance with the methods and systems disclosed herein.

In some examples, the selecting at block 1212 is based on user input. In other words, the method 1200 comprises enabling a user to select the first workflow. In other examples, the first workflow may be selected automatically, without user input. In some examples, the method 1200 comprises automatically identifying the previously-generated radiotherapy treatment plan, replacing at least part of the scheduled radiotherapy treatment plan with the previously-generated radiotherapy treatment plan, and determining and displaying information related to dose delivery for the previously-generated radiotherapy treatment plan.

In some examples, the method 1200 is performed during a radiotherapy fraction session.

In some examples, the method further comprises enabling selection, by a user, of one of at least the first workflow path and a second workflow path, wherein the second workflow path comprises calculating an adapted radiotherapy plan based on the discrepancy. Advantageously, the user is able to adapt a scheduled radiotherapy treatment plan either by using a previously-generated radiotherapy treatment plan, thereby saving time, or by calculating a new radiotherapy treatment plan, thereby enabling more detailed adaptation.

In the second workflow path, either or both of an adapt-to-position or adapt-to-shape approach may be used, dependent upon the particular discrepancy for the ROI. The second workflow path may comprise calculating an adapted radiotherapy treatment plan in accordance with any of the approaches disclosed herein as well as approaches known to the skilled person.

The method 1200 may also comprise one or more blocks of other methods disclosed herein, such as of the workflow 200 of FIG. 2 or the method 300 of FIG. 3. For example, the second workflow may include performing block 252 and/or 254 of the workflow 200 of FIG. 2, along with subsequent blocks of that workflow, in order to adapt the scheduled treatment plan. In other examples, the steps of those blocks may be used to adapt the previously-generated radiotherapy plan. In another example, the second workflow may include block(s) 206 onwards of the workflow 200 of FIG. 2 to assess plan quality. The method 1200 may in some examples include any one or more of blocks 306 to 312 of the method 300 of FIG. 3 in relation to at least one of the scheduled radiotherapy treatment plan and the previously-generated radiotherapy treatment plan. In some examples, after the previously-generated radiotherapy treatment plan is identified, it may be used as the scheduled radiotherapy treatment plan is within the examples of FIGS. 2 and 3. For example, block 308 of FIG. 3 may be performed on the previously-generated radiotherapy treatment plan in order to determine whether delivering the previously-generated radiotherapy treatment plan would meet at least one pre-defined treatment plan quality criterion, and to enable selection, by a user, of delivering the previously-generated radiotherapy treatment plan or adapting the previously-generated radiotherapy treatment plan. Further examples are set out below.

In some examples, the method 1200 further comprises determining, based on the comparing the obtained at least one image with the at least one reference image, a positional adjustment of an adjustable patient support that would move a current position of the ROI to a second position in which the ROI is more closely aligned with the reference position for the ROI.

In some examples, the method 1200 further comprises displaying a visual indicator of whether delivering the scheduled radiotherapy treatment plan with the ROI at the second position would meet at least one pre-defined treatment plan quality criterion. In some examples, the enabling step of the method 1200 further comprises enabling selection, by a user, of a third workflow path, wherein the third workflow path comprises executing the positional adjustment and delivering treatment according to the scheduled radiotherapy treatment plan.

In some examples, calculating the adapted radiotherapy plan comprises calculating beam shaping parameters to be used in delivering treatment, as described elsewhere herein.

In some examples, the adapted radiotherapy plan is calculated based on one or more parameters corresponding to at least one pre-defined treatment plan quality criterion that would not be met by delivering the scheduled radiotherapy treatment plan with the ROI at the second position.

In some examples, the second workflow path further comprises providing the adapted radiotherapy plan for remote review.

Also provided herein is a system for use in the radiotherapy workflow 200 and/or another radiotherapy workflow. The system is arranged to perform the methods disclosed herein, such as the methods 300 of FIG. 3 and/or the method(s) 1200 of FIG. 12, and comprises at least one processor and one or more non-transitory computer-readable media storing computer-executable instructions that, when executed by the one or more processors, cause the one or more processors to perform any one or more of the methods disclosed herein. The system may comprise a radiotherapy device including radiotherapy delivery apparatus, such as those of FIGS. 1 and 8. Accordingly, the system may comprise a computing system, image acquisition device, treatment device, input device and/or output device like those of FIG. 8.

FIG. 8 illustrates a block diagram of one implementation of a radiotherapy system 800. The radiotherapy system 800 comprises a computing system 810 within which a set of instructions, for causing the computing system 810 to perform any one or more of the methods discussed herein, may be executed.

The computing system 810 shall be taken to include any number or collection of machines, e.g. computing device(s), that individually or jointly execute a set (or multiple sets) of instructions to perform any one or more of the methods discussed herein. That is, hardware and/or software may be provided in a single computing device, or distributed across a plurality of computing devices in the computing system. In some implementations, one or more elements of the computing system may be connected (e.g., networked) to other machines, for example in a Local Area Network (LAN), an intranet, an extranet, or the Internet. One or more elements of the computing system may operate in the capacity of a server or a client machine in a client-server network environment, or as a peer machine in a peer-to-peer (or distributed) network environment. One or more elements of the computing system may be a personal computer (PC), a tablet computer, a set-top box (STB), a Personal Digital Assistant (PDA), a cellular telephone, a web appliance, a server, a network router, switch or bridge, or any machine capable of executing a set of instructions (sequential or otherwise) that specify actions to be taken by that machine.

The computing system 810 includes controller circuitry 811 and a memory 813 (e.g., read-only memory (ROM), flash memory, dynamic random access memory (DRAM) such as synchronous DRAM (SDRAM) or Rambus DRAM (RDRAM), etc.). The memory 813 may comprise a static memory (e.g., flash memory, static random access memory (SRAM), etc.), and/or a secondary memory (e.g., a data storage device), which communicate with each other via a bus (not shown).

Controller circuitry 811 represents one or more general-purpose processors such as a microprocessor, central processing unit, accelerated processing units, or the like. More particularly, the controller circuitry 811 may comprise a complex instruction set computing (CISC) microprocessor, reduced instruction set computing (RISC) microprocessor, very long instruction word (VLIW) microprocessor, processor implementing other instruction sets, or processors implementing a combination of instruction sets. Controller circuitry 811 may also include one or more special-purpose processing devices such as an application specific integrated circuit (ASIC), a field programmable gate array (FPGA), a digital signal processor (DSP), network processor, or the like. One or more processors of the controller circuitry may have a multicore design. Controller circuitry 811 is configured to execute the processing logic for performing the operations and steps discussed herein.

The computing system 810 may further include a network interface circuitry 818. The computing system 810 may be communicatively coupled to an input device 820 and/or an output device 830, via input/output circuitry 817. In some implementations, the input device 820 and/or the output device 830 may be elements of the computing system 810. The input device 820 may include an alphanumeric input device (e.g., a keyboard or touchscreen), a cursor control device (e.g., a mouse or touchscreen), an audio device such as a microphone, and/or a haptic input device. The output device 830 may include an audio device such as a speaker, a video display unit (e.g., a liquid crystal display (LCD) or a cathode ray tube (CRT)), and/or a haptic output device. In some implementations, the input device 820 and the output device 830 may be provided as a single device, or as separate devices.

In some implementations, the computing system 810 may comprise image processing circuitry 819. Image processing circuitry 819 may be configured to process image data 880 (e.g. images, or imaging data), such as medical images obtained from one or more imaging data sources, a treatment device 850 and/or an image acquisition device 840. Image processing circuitry 819 may be configured to process, or pre-process, image data. For example, image processing circuitry 819 may convert received image data into a particular format, size, resolution or the like. In some implementations, image processing circuitry 819 may be combined with controller circuitry 811.

In some implementations, the radiotherapy system 800 may further comprise an image acquisition device 840 and/or a treatment device 850, such as those disclosed herein in the examples of FIGS. 1 to 3. The image acquisition device 840 and the treatment device 850 may be provided as a single device. In some implementations, treatment device 850 is configured to perform imaging, for example in addition to providing treatment and/or during treatment. The treatment device 850 comprises the main radiation delivery components of the radiotherapy system, such as the linac.

Image acquisition device 840 may be configured to perform positron emission tomography (PET), computed tomography (CT), magnetic resonance imaging (MRI), or other suitable imaging techniques.

Image acquisition device 840 may be configured to output image data 880, which may be accessed by computing system 810. Treatment device 850 may be configured to output treatment data 860, which may be accessed by computing system 810.

Computing system 810 may be configured to access or obtain treatment data 860, planning data 870 and/or image data 880. Treatment data 860 may be obtained from an internal data source (e.g. from memory 813) or from an external data source, such as treatment device 850 or an external database. Planning data 870 may be obtained from memory 813 and/or from an external source, such as a planning database. Planning data 870 may comprise information obtained from one or more of the image acquisition device 840 and the treatment device 850. Accordingly, computing system 810 is arranged to implement the methods disclosed herein relating to handling treatment plans and treatment quality criteria.

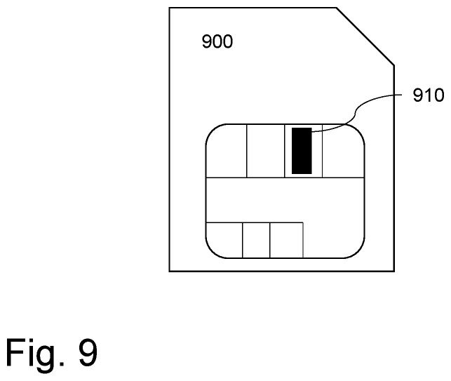

The various methods described above may be implemented by a computer program. The computer program may include computer code (e.g. instructions) 910 arranged to instruct a computer to perform the functions of one or more of the various methods described above. The steps of the methods described above may be performed in any suitable order. The computer program and/or the code 910 for performing such methods may be provided to an apparatus, such as a computer, on one or more computer readable media or, more generally, a computer program product 900)), depicted in FIG. 9. The computer readable media may be transitory or non-transitory. The one or more computer readable media 900 could be, for example, an electronic, magnetic, optical, electromagnetic, infrared, or semiconductor system, or a propagation medium for data transmission, for example for downloading the code over the Internet. Alternatively, the one or more computer readable media could take the form of one or more physical computer readable media such as semiconductor or solid state memory, magnetic tape, a removable computer diskette, a random access memory (RAM), a read-only memory (ROM), a rigid magnetic disc, and an optical disk, such as a CD-ROM, CD-R/W or DVD. The instructions 910 may also reside, completely or at least partially, within the memory 813 and/or within the controller circuitry 811 during execution thereof by the computing system 810, the memory 813 and the controller circuitry 811 also constituting computer-readable storage media.

In an implementation, the modules, components and other features described herein can be implemented as discrete components or integrated in the functionality of hardware components such as ASICS, FPGAS, DSPs or similar devices.

A “hardware component” is a tangible (e.g., non-transitory) physical component (e.g., a set of one or more processors) capable of performing certain operations and may be configured or arranged in a certain physical manner. A hardware component may include dedicated circuitry or logic that is permanently configured to perform certain operations. A hardware component may comprise a special-purpose processor, such as an FPGA or an ASIC. A hardware component may also include programmable logic or circuitry that is temporarily configured by software to perform certain operations.

In addition, the modules and components can be implemented as firmware or functional circuitry within hardware devices. Further, the modules and components can be implemented in any combination of hardware devices and software components, or only in software (e.g., code stored or otherwise embodied in a machine-readable medium or in a transmission medium).

Unless specifically stated otherwise, as apparent from the following discussion, it is appreciated that throughout the description, discussions utilizing terms such as “receiving”, “determining”, “comparing”, “enabling”, “maintaining”, “identifying”, “calculating”, “displaying”, or the like, refer to the actions and processes of a computer system, or similar electronic computing device, that manipulates and transforms data represented as physical (electronic) quantities within the computer system's registers and memories into other data similarly represented as physical quantities within the computer system memories or registers or other such information storage, transmission or display devices.

It is to be understood that the above description is intended to be illustrative, and not restrictive. Many other implementations will be apparent to those of skill in the art upon reading and understanding the above description. Although the present disclosure has been described with reference to specific example implementations, it will be recognized that the disclosure is not limited to the implementations described, but can be practiced with modification and alteration within the spirit and scope of the appended claims. Accordingly, the specification and drawings are to be regarded in an illustrative sense rather than a restrictive sense. The scope of the disclosure should, therefore, be determined with reference to the appended claims, along with the full scope of equivalents to which such claims are entitled.

1. A computer-implemented method for use in a radiotherapy workflow, comprising:
obtaining at least one image of a patient, wherein the at least one image of the patient indicates a current position for a region of interest, ROI, of the patient;
comparing the obtained at least one image with at least one reference image associated with a scheduled radiotherapy treatment plan for the patient, wherein the at least one reference image indicates a reference position for the ROI of the patient;
determining, based on the comparing, a discrepancy between the current position for the ROI and the reference position for the ROI;
determining a structural characteristic of the obtained at least one image;
based on the determined structural characteristic, identifying a previously-generated radiotherapy treatment plan; and
selecting a first workflow path, wherein the first workflow path comprises replacing at least part of the scheduled radiotherapy treatment plan with at least part of the previously-generated radiotherapy treatment plan.

2. The method of claim 1, wherein identifying the previously-generated radiotherapy treatment plan comprises retrieving the previously-generated radiotherapy treatment plan from a repository comprising a plurality of previously-generated radiotherapy treatment plans.

3. The method of claim 1, wherein identifying the previously-generated radiotherapy treatment plan comprises comparing the determined structural characteristic of the obtained at least one image with a structural characteristic associated with the previously-generated radiotherapy treatment plan.

4. The method of claim 1, wherein identifying the previously-generated radiotherapy treatment plan comprises determining a similarity between the determined structural characteristic of the obtained at least one image and a structural characteristic associated with the previously-generated radiotherapy treatment plan.

5. The method of claim 1, wherein identifying the previously-generated radiotherapy treatment plan further comprises determining a Dice similarity coefficient for the obtained at least one image and an image associated with the previously-generated radiotherapy treatment plan.

6. The method of claim 1, wherein the determined structural characteristic is representative of at least one of: a region of interest, a treatment target, an organ-at-risk, a Hausdorff distance, a volume, or an overlap volume histogram.

7. The method of claim 1, wherein the determined structural characteristic is representative of a plurality of anatomical structures.

8. The method of claim 1, further comprising:
displaying information representative of dose characteristics associated with the identified previously-generated radiotherapy plan.

9. The method of claim 1, wherein the method is performed during a radiotherapy fraction session.

10. The method of claim 1, wherein the discrepancy comprises at least one of: a difference in position for the ROI, a difference in location for the ROI, or a difference in shape for the ROI.

11. The method of claim 1, further comprising:
determining that the previously-generated radiotherapy treatment plan meets at least one pre-defined treatment plan quality criterion, the at least one pre-defined treatment plan quality criterion comprising one or more of: a minimum target dose threshold, a maximum organ at risk dose threshold, or the ROI exhibiting a threshold degree of similarity between the at least one obtained image and the at least one reference image.

12. The method of claim 1, wherein the first workflow path further comprises determining that the identified previously-generated radiotherapy treatment plan is suitable for delivering a prescribed dose associated with the scheduled radiotherapy treatment plan.

13. The method of claim 1, wherein the selecting is based on user input.

14. The method of claim 1, wherein the method further comprises:
enabling selection, by a user, of one of at least the first workflow path and a second workflow path, wherein the second workflow path comprises calculating an adapted radiotherapy plan based on the discrepancy.

15. The method of claim 14, the method further comprising:
determining, based on the comparing the obtained at least one image with the at least one reference image, a positional adjustment of an adjustable patient support to move a current position of the ROI to a second position in which the ROI is more closely aligned with the reference position for the ROL

16. The method of claim 15, wherein at least one of:
the method further comprises displaying a visual indicator of whether delivering the scheduled radiotherapy treatment plan with the ROI at the second position meets at least one pre-defined treatment plan quality criterion; or
the method further comprises enabling selection, by a user, of a third workflow path, wherein the third workflow path comprises executing the positional adjustment and delivering treatment according to the scheduled radiotherapy treatment plan.

17. The method of claim 15, wherein calculating the adapted radiotherapy plan comprises calculating beam shaping parameters to be used in delivering treatment.

18. The method of claim 15, wherein the adapted radiotherapy plan is calculated based on one or more parameters corresponding to at least one pre-defined treatment plan quality criterion that would not be met by delivering the scheduled radiotherapy treatment plan with the ROI at the second position.

19. The method of claim 15, wherein the second workflow path further comprises providing the adapted radiotherapy plan for remote review.

20. A system for use in a radiotherapy workflow, the system comprising:
at least one processor; and
one or more non-transitory computer-readable media storing computer-executable instructions that, when executed by the at least one processor, cause the at least one processor to perform operations comprising:
obtaining at least one image of a patient, wherein the at least one image of the patient indicates a current position for a region of interest, ROI, of the patient;
comparing the obtained at least one image with at least one reference image associated with a scheduled radiotherapy treatment plan for the patient, wherein the at least one reference image indicates a reference position for the ROI of the patient;
determining, based on the comparing, a discrepancy between the current position for the ROI and the reference position for the ROI;
determining a structural characteristic of the obtained at least one image;
based on the determined structural characteristic, identifying a previously-generated radiotherapy treatment plan; and
selecting a first workflow path, wherein the first workflow path comprises replacing at least part of the scheduled radiotherapy treatment plan with at least part of the previously-generated radiotherapy treatment plan.

21. (canceled)

22. A non-transitory computer-readable medium containing instructions that, when executed by a processor, cause the processor to perform operations comprising:
obtaining at least one image of a patient, wherein the at least one image of the patient indicates a current position for a region of interest, ROI, of the patient;
comparing the obtained at least one image with at least one reference image associated with a scheduled radiotherapy treatment plan for the patient, wherein the at least one reference image indicates a reference position for the ROI of the patient:
determining, based on the comparing, a discrepancy between the current position for the ROI and the reference position for the ROI:
determining a structural characteristic of the obtained at least one image;
based on the determined structural characteristic, identifying a previously-generated radiotherapy treatment plan; and
selecting a first workflow path, wherein the first workflow path comprises replacing at least part of the scheduled radiotherapy treatment plan with at least part of the previously-generated radiotherapy treatment plan.
